(19) United States
(12) Patent Application Publication
(10) Pub. No.: US 2024/0041338 A1
(43) Pub. Date: Feb. 8, 2024

(54) CUFF COVER

(71) Applicant: OMRON HEALTHCARE Co., Ltd., Kyoto (JP)

(72) Inventors: Takeshi KUBO, Kyoto (JP); Kentaro MORI, Kyoto (JP); Masaki HARADA, Kyoto (JP)

(21) Appl. No.: 18/487,747

(22) Filed: Oct. 16, 2023

Related U.S. Application Data
(63) Continuation of application No. PCT/JP2022/019550, filed on May 6, 2022.

(30) Foreign Application Priority Data
May 28, 2021 (JP) 2021-090313

(51) Int. Cl. A61B 5/022; A61B 5/256; A61B 5/282

(52) U.S. Cl. CPC A61B 5/02233; A61B 5/256; A61B 5/282

(57) ABSTRACT

A cuff cover configured to cover a cuff structure in a biological information measurement device, the biological information measurement device including the cuff structure including one or more cuffs and electrodes, and an attachment unit, the biological information measurement device configured to measure blood pressure value and an electrocardiographic waveform of a human body attached to the human body by the attachment unit, the cuff cover including a covering portion formed of a non-conductive material, the covering portion configured to cover the cuff structure, a mounting portion configured to mount the covering portion to the cuff structure, and an energization assisting unit provided in the covering portion, the energization assisting unit configured to assist energization between each of the one or more electrodes included in the cuff structure covered with the covering portion and the human body when the biological information measurement device is attached to the human body.

### CROSS-REFERENCE TO RELATED APPLICATIONS

This application is the U.S. national stage application filed pursuant to 35 U.S.C. 365(c) and 120 as a continuation of International Patent Application No. PCT/JP2022/019550, filed May 6, 2022, which application claims priority to Japanese Patent Application No. 2021-090313, filed 28 May 2021, which applications are incorporated herein by reference in their entireties.

### TECHNICAL FIELD

The present invention belongs to the technical field related to healthcare, and particularly relates to a cuff cover to be used for a biological information measurement device.

### BACKGROUND ART

In recent years, it has been common that an individual measures information (hereinafter, also referred to as biological information) related to the body and health of the individual such as a blood pressure value and an electrocardiographic waveform on a daily basis by himself/herself by using a measurement device and utilizes the measurement result for health management. For this reason, the demand for devices where importance is placed on portability has been increasing, many portable measurement devices have been proposed, and portable devices that can measure both the blood pressure value and the electrocardiographic waveform have also been proposed (for example, Patent Document 1).

Patent Document 1 discloses a portable cardiovascular measurement device including a cuff for blood pressure measurement and an electrode for electrocardiographic measurement. According to the invention, a user can measure a blood pressure value and an electrocardiographic waveform at a freely selected timing by carrying the device.

Incidentally, in such a device, it is necessary to mount a cover in order to prevent a cuff (air bag) that compresses a human body when a blood pressure is measured from being contaminated or deteriorated due to direct contact with the human body, and such a cuff cover is obtained by a known technique (for example, Patent Document 2).

The cuff cover is detachable from the cuff and needs to be made of a material having stretchability such as cloth that does not interfere with expansion and contraction of the cuff. Patent Document 2 also proposes a bag-shaped cuff cover formed of such a cloth material having stretchability.

### CITATION LIST

### Patent Literature

Patent Document 1: JP 2014-36843 A
Patent Document 2: JP 2020-103645 A

### Technical Problem

### SUMMARY OF INVENTION

However, when a cuff cover made of a cloth material as disclosed in Patent Document 2 is used in a device capable of measuring both an electrocardiographic waveform and a blood pressure value, there is a problem that electrocardiographic measurement electrodes installed on a cuff are insulated from a human body at attached portions, and thus, an electrocardiogram cannot be measured.

In view of the problems described above, an object of the present invention is to provide a cuff cover suitable for a biological information measurement device including a cuff for blood pressure measurement and electrodes for electrocardiographic measurement.

### Solution to Problem

In order to solve the above problems, a cuff cover according to the present invention is configured to cover a cuff structure in a biological information measurement device, the biological information measurement device including the cuff structure including one or more cuffs and one or more electrodes, and an attachment unit, the biological information measurement device being configured to measure a blood pressure value and an electrocardiographic waveform of a human body in a state of being attached to the human body by the attachment unit, the cuff cover including:
a covering portion formed of a non-conductive material, the covering portion being configured to cover the cuff structure,
a mounting portion configured to mount the covering portion to the cuff structure, and an energization assisting unit provided in the covering portion, the energization assisting unit being configured to assist energization between each of the one or more electrodes included in the cuff structure covered with the covering portion and the human body when the biological information measurement device is attached to the human body.

According to the cuff cover having such a configuration, a situation in which the one or more electrodes are insulated from the human body to be measured by the cover, and the electrocardiographic measurement cannot be performed can be prevented, which allows the cuff cover suitable for the biological information measurement device capable of measuring both the blood pressure value and the electrocardiographic waveform to be provided. Note that the mounting portion may include various materials according to a form of the cuff cover. For example, an adhesive, a double-sided tape, a rubber band, a hook-and-loop fastener, and snap buttons, and the like can be adopted.

In addition, the energization assisting unit may be an opening portion provided in the covering portion in a manner that each of the one or more electrodes is configured to be exposed. With such a configuration, since the electrodes for electrocardiographic measurement can be brought into direct contact with the human body, the electrocardiographic waveform can be measured with high accuracy.

In addition, in a state in which the cuff structure is covered with the covering portion, the energization assisting unit may be a conductive portion configured to cover a portion including each of the one or more electrodes included in the cuff structure. Note that the conductive portion may be formed by weaving conductive fibers into the covering portion, or may be formed by covering at least a part of an opening provided in the covering portion with a lid-shaped member made of a conductive material. According to such a configuration, since the cuff structure including the one or more electrodes can be covered, the cuff structure can be more reliably protected, and the one or more electrodes and the human body can be electrically connected through the conductive portion.

In addition, the covering portion may be formed in a bag shape capable of accommodating the cuff structure, and may include an accommodation port that is an opening configured to accommodate the cuff structure in the covering portion. With such a structure, since the cuff structure can be accommodated in the covering portion having the bag shape to cover the entire cuff structure, a function of protecting the cuff structure can be further improved.

In addition, the covering portion may be formed of a material having stretchability, and may include an inner covering portion configured to be brought into contact with the human body in a state in which the cuff structure is accommodated, the inner covering portion being provided with the energization assisting unit, and an outer covering portion configured to be opposed to the inner covering portion with the cuff structure interposed between the outer covering portion and the inner covering portion in the state in which the cuff structure is accommodated,
the accommodation port may be provided in the outer covering portion, may be an opening having an area smaller than an area of the cuff structure in a plan view of the cuff structure, and may be expandable to be larger than the area of the cuff structure due to the stretchability of the material when the cuff structure is accommodated, and the mounting portion may be the accommodation port in the covering portion.

With such a configuration, the accommodation port provided in the covering portion and configured to be expandable can be used as the mounting portion for the cuff cover without separately providing a unit for mounting the cuff cover, which allows the number of components of a product to be reduced.

In addition, the covering portion may include a plurality of the accommodation ports, may be configured to accommodate a plurality of structures including the cuff structure in the covering portion from different accommodation ports, and may be provided with a fixing portion configured to fix the inner covering portion and the outer covering portion at a position between one of the plurality of the accommodation ports and another of the plurality of accommodation ports. With such a configuration, in a state in which the cuff structure is accommodated in the covering portion, “floating” of the covering portion can be prevented in the vicinity of the fixing portion, and positional deviation between each of the one or more electrodes of the cuff structure accommodated in the covering portion and a conduction assisting portion in the covering portion can be suppressed.

Note that the configurations described above can be combined with one another to constitute the present invention unless the combination leads to contradiction.

### Advantageous Effects of Invention

According to the present invention, the cuff cover suitable for the biological information measurement device including the cuff for blood pressure measurement and the electrodes for electrocardiographic measurement can be provided.

### BRIEF DESCRIPTION OF DRAWINGS

FIG. 1(A) is a schematic plan view illustrating an appearance of a cuff cover according to Embodiment 1. FIG. 1(B) is a schematic bottom view illustrating the appearance of the cuff cover according to Embodiment 1.

FIG. 2 is a schematic plan view illustrating an appearance of a biological information measurement device for which the cuff cover according to Embodiment 1 is used.

FIG. 3(A) is a schematic bottom view illustrating the appearance of the biological information measurement device for which the cuff cover according to Embodiment 1 is used. FIG. 3(B) is a schematic side view illustrating the appearance of the biological information measurement device for which the cuff cover according to Embodiment 1 is used.

FIG. 4(A) is a first diagram illustrating a state in which the cuff cover according to Embodiment 1 is mounted to the biological information measurement device. FIG. 4(B) is a second diagram illustrating the state in which the cuff cover according to Embodiment 1 is mounted to the biological information measurement device.

FIG. 5(A) is a third diagram illustrating the state in which the cuff cover according to Embodiment 1 is mounted to the biological information measurement device. FIG. 5(B) is a fourth diagram illustrating the state in which the cuff cover according to Embodiment 1 is mounted to the biological information measurement device.

FIG. 6 is an explanatory diagram illustrating arrangement relationships among constituent elements when the biological information measurement device to which the cuff cover according to Embodiment 1 is mounted is attached to a wrist.

FIG. 7(A) is a first diagram illustrating a conduction assisting portion of a cuff cover according to Modified Example 1 of Embodiment 1. FIG. 7(B) is a second diagram illustrating the conduction assisting portion of the cuff cover according to Modified Example 1 of Embodiment 1.

FIG. 8(A) is a first diagram illustrating a conduction assisting portion of a cuff cover according to Modified Example 2 of Embodiment 1. FIG. 8(B) is a second diagram illustrating the conduction assisting portion of the cuff cover according to Modified Example 2 of Embodiment 1.

FIG. 9(A) is a schematic plan view illustrating an appearance of a cuff cover according to Modified Example 3 of Embodiment 1. FIG. 9(B) is an explanatory diagram illustrating a state in which the cuff cover according to Modified Example 3 of Embodiment 1 is mounted to a biological information measurement device.

FIG. 10(A) is a schematic plan view illustrating an appearance of a cuff cover according to Embodiment 2. FIG. 10(B) is a schematic bottom view illustrating the appearance of the cuff cover according to Embodiment 2.

FIG. 11(A) is a schematic bottom view illustrating an appearance of a biological information measurement device for which the cuff cover according to Embodiment 2 is used. FIG. 11(B) is a schematic side view illustrating the appearance of the biological information measurement device for which the cuff cover according to Embodiment 2 is used.

### DESCRIPTION OF EMBODIMENTS

### Embodiment 1

Embodiments of the present invention will be specifically described below with reference to the drawings. It should be noted that the dimensions, material, shape, relative arrangement and the like of the constituent components described in the embodiments are not intended to limit the scope of this invention to them alone, unless otherwise stated.

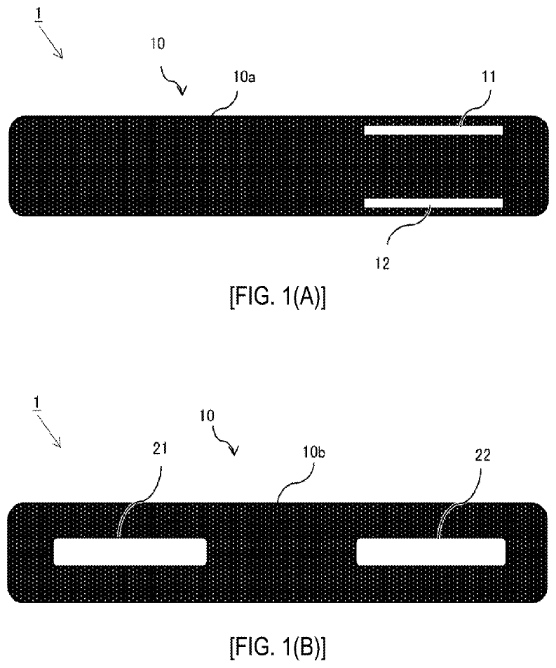

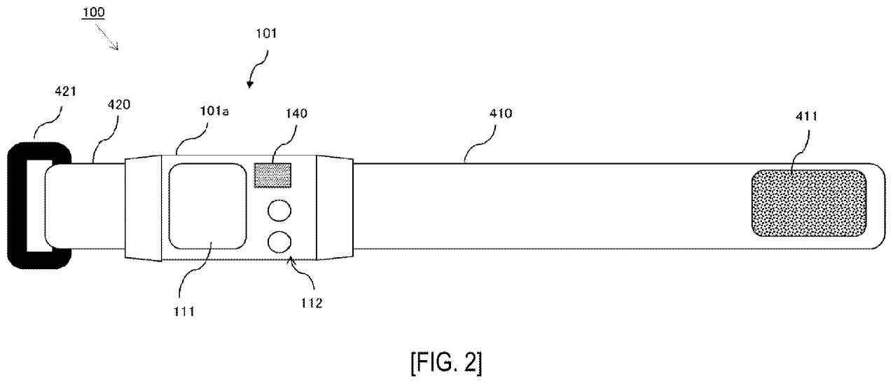

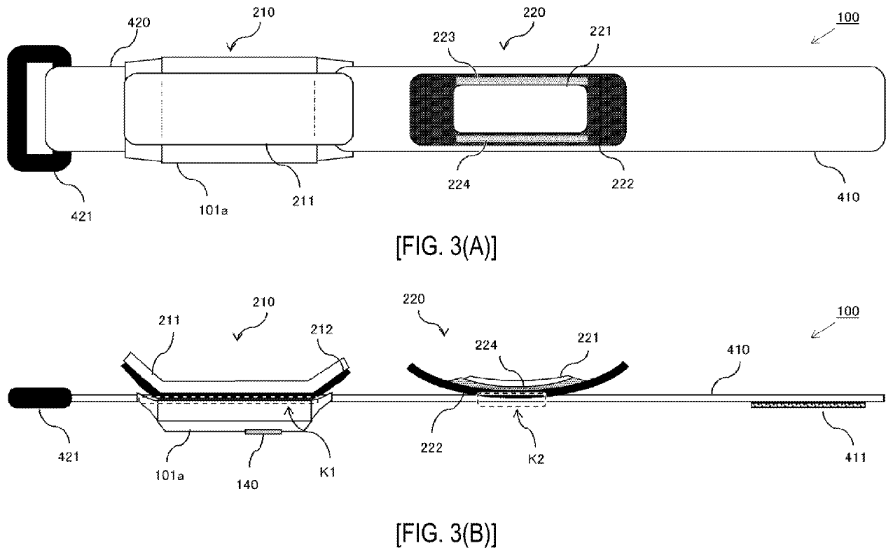

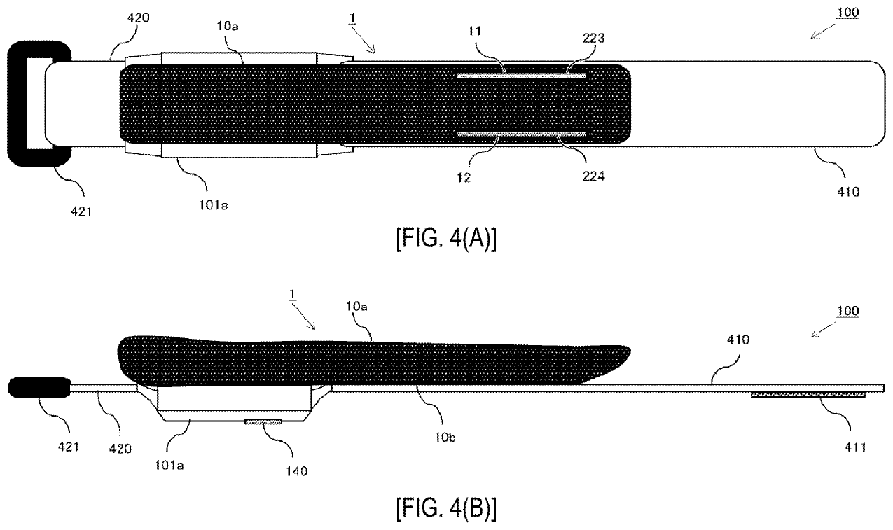

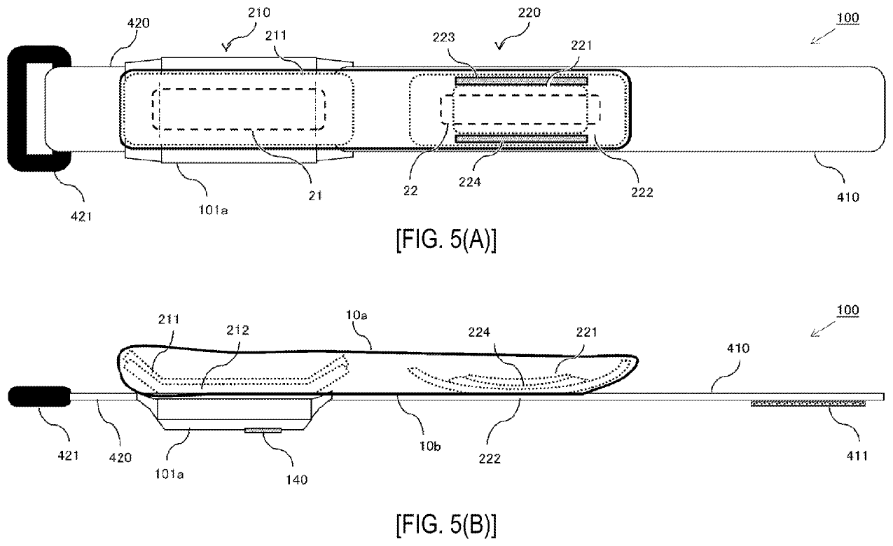

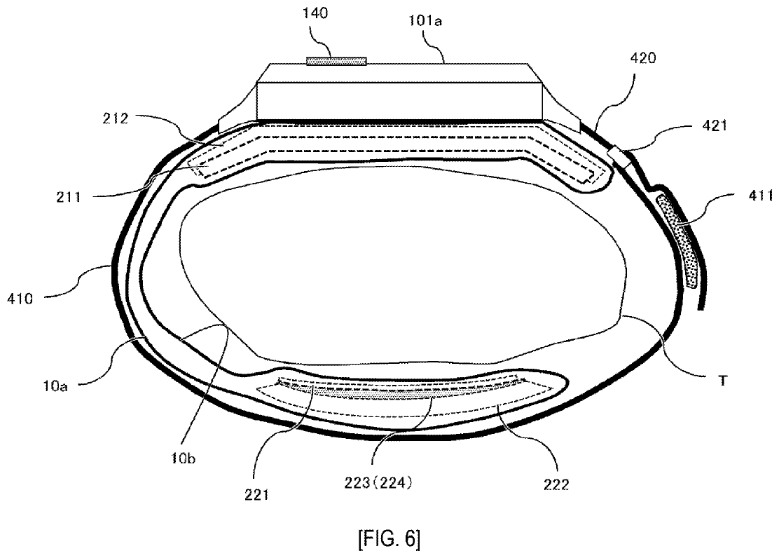

FIGS. 1(A) and 1(B) are views illustrating an appearance of a cuff cover 1 according to the present embodiment, and FIGS. 2, and 3(A) and 3(B) are schematic views illustrating an appearance configuration of a biological information measurement device 100 for which the cuff cover 1 is used. Additionally, FIGS. 4(A) and 4(B), and FIGS. 5(A) and 5(B) are explanatory diagrams illustrating a state in which the cuff cover 1 is mounted to the biological information measurement device 100, and FIG. 6 is an explanatory view illustrating arrangement relationships among constituent elements when the biological information measurement device 100 to which the cuff cover 1 is mounted is attached to a wrist.

### Configuration of Cuff Cover

FIG. 1(A) is a schematic plan view illustrating the appearance of the cuff cover 1, and FIG. 1(B) is a schematic bottom view illustrating the appearance of the cuff cover 1. As illustrated in FIGS. 1(A) and 1(B), the cuff cover 1 according to the present embodiment has a substantially rectangular shape having a longitudinal direction, and is constituted by a covering portion 10 formed in a bag shape capable of accommodating cuff structures (in the present embodiment, a pressing cuff portion 210 and a sensing cuff portion 220, which will be described later) of the biological information measurement device 100 therein as will be described later. The covering portion 10 is formed of a stretchable cloth material, but a forming method thereof is not particularly limited. The covering portion 10 may be formed by knitting, or may be formed by joining a plurality of cloth materials by sewing, thermal welding, or the like.

Here, the stretchability of the covering portion 10 does not interfere with blood pressure measurement (which will be described later) by the biological information measurement device 100, and specifically, the covering portion 10 has stretchability to such an extent that expansion and contraction of a pressing cuff 211 and a sensing cuff 221, which will be described later, are not inhibited.

In addition, the covering portion 10 includes an inner covering portion 10a on a side to be brought into contact with a wrist of a measurement target when the biological information measurement device 100 is attached to the wrist in a state in which the cuff structure is accommodated, and an outer covering portion 10b on a side to be opposed to the inner covering portion 10a with the cuff structure interposed therebetween. Note that in the present specification, when the biological information measurement device 100 is attached to the wrist, the side to be brought into contact with the wrist is referred to as an inner side, and the opposite side thereto is referred to as an outer side in the following description.

The inner covering portion 10a is provided with opening portions 11 and 12 for electrodes configured to expose electrodes of the cuff structure accommodated in the covering portion as will be described later. That is, in the present embodiment, each of the opening portions 11 and 12 for electrodes corresponds to the energization assisting unit. In addition, the outer covering portion 10b is provided with accommodation ports 21 and 22 configured to accommodate the cuff structures inside the covering portion 10.

### Configuration of Biological Information Measurement Device

FIG. 2 is a schematic plan view illustrating an appearance of the biological information measurement device 100 to which the cuff cover 1 is mounted. In addition, FIG. 3(A) is a schematic view illustrating a bottom surface of the biological information measurement device 100, and FIG. 3(B) is a schematic view illustrating a side surface of the biological information measurement device 100.

As illustrated in FIGS. 2 and 3(A) and 3(B), the biological information measurement device 100 generally includes a main body unit 101, the pressing cuff portion 210 and the sensing cuff portion 220 (cuff structures), and a belt portion 400, and can measure a blood pressure value and an electrocardiographic waveform in a state in which the biological information measurement device 100 is attached to a wrist of a human body. The belt portion 400 includes a first belt portion 410 including a hook-and-loop fastener 411 including a hook, and a second belt portion 420 including a belt loop 421. When the biological information measurement device 100 is attached, the first belt portion 410 is wound around the wrist and inserted through the belt loop 421, and the hook-and-loop fastener 411 is stuck to a freely selected position of the first belt portion 410 in which a loop with which a hook engages is formed, thereby fixing the biological information measurement device 100.

### Main Body Unit

The main body unit 101 includes a housing 101a, a display unit 111, an operation unit 112, and a first electrode 140. In addition, although not illustrated, the main body unit 101 includes a power supply unit, a control unit, a circuit electrically connected to the electrodes, which will be described below, a pump configured to pressure-feed air to the cuff, a fluid flow path, an exhaust valve, and the like. Note that a battery configured to supply power that is required to drive the device may be used as a power source, and may be, for example, a secondary battery such as a lithium ion battery, or a primary battery.

In addition, the control unit controls the biological information measurement device 100, and also performs calculation for measuring a blood pressure value and an electrocardiographic waveform of a measurement target based on information acquired from the cuff structure by controlling the above-described pump and the like. The control unit may be constituted by, for example, a central processing unit (CPU) and the like, and may include a storage device such as a random access memory (RAM).

The display unit 111 may include a display device such as a liquid crystal display, and may be provided with an LED indicator or the like. The operation unit 112 may include various operation buttons such as a power button, for example, but may be configured such that the display unit 111 and the operation unit 112 are integrated, like a touch panel display.

The first electrode 140 is an electrode configured to measure an electrocardiographic waveform of a wearer of the biological information measurement device 100, and is electrically connected to the electronic circuit of the main body unit 101. Bringing a finger or the like of a hand on which the biological information measurement device 100 is not attached into contact with the first electrode 140 allows an electrocardiographic waveform to be measured based on a potential difference between the first electrode 140 and a second electrode 223, which will be described later.

### Cuff Structure

As illustrated in FIG. 3(B), the pressing cuff portion 210 that is one of the cuff structures is fixed to the housing 101a of the main body by a fixing portion K1, and includes the pressing cuff 211 that expands by air sent from the pump to press the wrist that is an attached portion, and a pressing cuff plate 212 that supports the pressing cuff 211.

As illustrated in FIGS. 3(A) and 3(B), the sensing cuff portion 220, which is the other cuff structure, is fixed to the first belt portion 410 by a fixing portion K2, and includes the sensing cuff 221, a back plate 222, the second electrode 223, and a third electrode 224. The sensing cuff 221 is a fluid bag configured to detect a pressure applied to a portion compressed by a pressing force of the pressing cuff 211, and the pressure applied to the compressed portion is measured by detecting an internal pressure with a pressure meter (not illustrated) in a state in which a small amount of air is contained in the sensing cuff 221. The back plate 222 is a flexible flat plate member disposed outside the sensing cuff 221, suppresses excessive bending of the sensing cuff 221 at the time of compression by the pressing cuff 211, and balances the pressure distribution in the sensing cuff 221.

The second electrode 223 and the third electrode 224 are electrodes disposed at both ends in a lateral direction of the sensing cuff 221 so as to be contactable with the surface of the human body. The second electrode 223 functions as an electrode for electrocardiographic waveform measurement, and the third electrode 224 functions as an electrode for noise removal (right foot electrode).

### Measurement of Biological Information

Biological information by the biological information measurement device 100 having the above-described configuration is measured in the following manner. First, the main body unit 101 is directed to the back side of a hand, the pressing cuff portion 210 is positioned on the back side of the wrist, the sensing cuff portion 220 is positioned on the palm side of the wrist, and the biological information measurement device 100 is fixed to the wrist by using the belt portion 400. Specifically, the first belt portion 410 is wound around the wrist, the first belt portion 410 is passed through the belt loop 421 of the second belt portion 420 and folded back, the hook-and-loop fastener 411 of the first belt portion 410 is stuck to a freely selected position of the first belt portion 410, and the biological information measurement device 100 is attached to the wrist.

Then, the operation unit 112 is operated to start measurement. Specifically, when air is injected into the pressing cuff 211, the wrist is pressed against the sensing cuff 221 side and (an artery of) the wrist is compressed to occlude the artery. After the blood flow is temporarily stopped in this way, air is gradually discharged from the pressing cuff 211 to restore the blood flow in the artery, and the pressure at that time is measured by the sensing cuff 221. That is, blood pressure is measured by a so-called oscillometric method.

Then, during the blood pressure measurement, when the wrist is pressed by the pressing cuff 211, the second electrode 223 and the third electrode 224 are in contact with (pressed against) the surface of the wrist. Accordingly, by touching the first electrode 140 provided on the housing 101a of the main body unit 101 with the fingers on the side on which the biological information measurement device 100 is not attached, an electrocardiographic waveform can be measured by a so-called I induction method based on a potential difference between the first electrode 140 and the second electrode 223.

### Form When Cuff Cover is Mounted

As described above, since the cuff comes into contact with a human body when biological information is measured by the biological information measurement device 100, the cuff cover 1 is mounted to the cuff structures to prevent the cuff structures themselves from being contaminated or deteriorated. FIG. 4(A) is a schematic view illustrating a bottom surface of the biological information measurement device 100 in a state in which the cuff cover 1 is mounted to the pressing cuff portion 210 and the sensing cuff portion 220, and FIG. 4(B) is a schematic view illustrating a side surface of the biological information measurement device 100 when the cuff cover 1 is mounted. Further, FIG. 5(A) is an explanatory diagram illustrating the cuff cover 1 in a transparent state in the diagram illustrated in FIGS. 4(A), and 5(B) is an explanatory diagram illustrating the cuff cover 1 in a transparent state in the diagram illustrated in FIG. 4(B). In addition, FIG. 6 is an explanatory diagram illustrating arrangement relationships among constituent elements in a state in which the biological information measurement device 100 to which the cuff cover 1 is mounted is attached to a wrist T.

As illustrated in FIG. 4(A), when the cuff cover 1 is mounted to the biological information measurement device 100, the pressing cuff portion 210 and the sensing cuff portion 220 are covered with the covering portion 10 except that the second electrode 223 and the third electrode 224 are exposed from the opening portions 11 and 12 for electrodes, respectively. Thus, as illustrated in FIG. 6, the other portions of the cuff structures can be covered without inhibiting the contact between each of the second electrode 223 and the third electrode 224 and the human body (that is, without insulating the electrodes from the human body), which can prevent the cuff structure from being contaminated.

The mounting of the cuff cover 1 to the biological information measurement device 100 is performed by accommodating the pressing cuff portion 210 and the sensing cuff portion 220 into the covering portion 10 through the accommodation port 21 and the accommodation port 22, respectively. Here, an area of the accommodation port 21 when the outer covering portion 10b is viewed in a plan view is smaller than an area of the pressing cuff portion 210 in a plan view, and a relationship between those of the accommodation port 22 and the sensing cuff portion 220 is also the same. For this reason, in order to accommodate the pressing cuff portion 210 and the sensing cuff portion 220 into the covering portion 10 from the accommodation ports 21 and 22, the accommodation ports 21 and 22 are pushed and spread (or the cloth around the accommodation ports is pulled) with fingers to expand the accommodation ports 21 and 22, and then, the pressing cuff portion 210 and the sensing cuff portion 220 are accommodated.

Since the covering portion 10 is formed of a material having stretchability, it is possible to expand the opening portion as described above. Also, when forces that expand the accommodation ports 21 and 22 are eliminated, the covering portion 10 tries to return to the original shape. In FIG. 5(A), the arrangement relationships among the constituent elements with the cuff cover 1 mounted to the biological information measurement device 100 illustrated in a transparent manner are illustrated, and the accommodation ports 21 and 22 are illustrated by broken lines with the pressing cuff portion 210 and the sensing cuff portion 220 illustrated in a transparent manner. As illustrated in FIG. 5(A), the accommodation ports 21 and 22 in the state returned from the expanded state (that is, in a normal state) are in a state of being hooked by the outer end portions of the pressing cuff portion 210 and the sensing cuff portion 220, respectively. That is, the cuff cover 1 is mounted to the biological information measurement device 100 by hooking the accommodation ports 21 and 22 on the pressing cuff portion 210 and the sensing cuff portion 220, respectively.

In addition, the cuff cover 1 mounted to the biological information measurement device 100 in this manner can be easily detached by expanding each of the accommodation ports 21 and 22 in the state of being hooked by the outer end portions of the pressing cuff portion 210 and the sensing cuff portion 220.

The cuff cover 1 according to the present embodiment having the above-described configuration can be easily detachably mounted to the cuff structures of the biological information measurement device including the cuff for blood pressure measurement and the electrodes for electrocardiographic measurement, and can simultaneously measure a blood pressure value and an electrocardiographic waveform with high accuracy because electrical conduction between the electrodes for electrocardiographic measurement and the human body is not inhibited.

### Modified Example 1

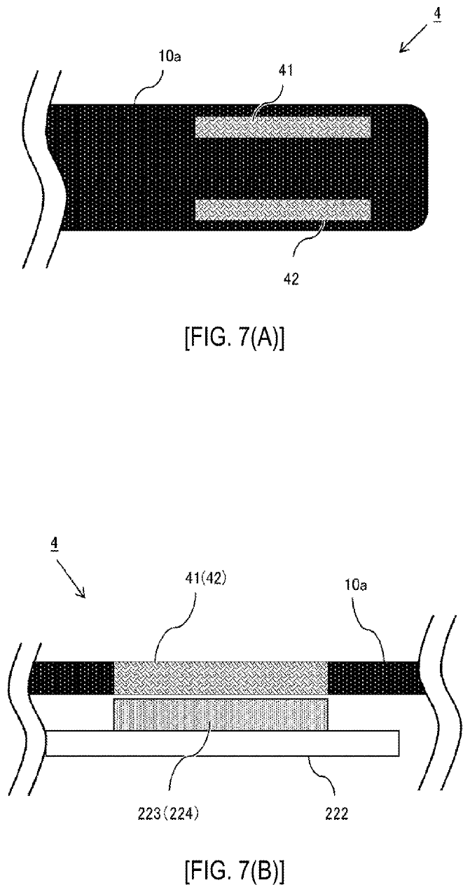

It should be noted that the cuff cover 1 according to Embodiment 1 can be modified in various ways. Such modified examples will be described below with reference to the accompanying drawings. FIGS. 7(A) and 7(B) are diagrams illustrating a cuff cover 4 according to Modified Example 1 of the cuff cover 1 according to Embodiment 1. FIG. 7(A) is a partial plan view of the inner covering portion 10a of the cuff cover 4 according to the present modified example, and FIG. 7(B) is an explanatory diagram illustrating conductive fiber portions 41 and 42 of the cuff cover 4 according to the present modified example.

Note that since the basic configuration of the cuff cover 4 according to the present modified example is the same as that of the cuff cover 1, such constituent elements are denoted by the same reference signs as those of the cuff cover 1, and redundant description is omitted. As illustrated in FIG. 7(A), the cuff cover 4 according to the present modified example includes the conductive fiber portions 41 and 42 instead of the opening portions 11 and 12 for electrodes in the inner covering portion 10a.

Conductive fibers (for example, desired metallic fibers) are woven into the conductive fiber portions 41 and 42, and as illustrated in FIG. 7(B), in the inner covering portion 10a, the conductive fiber portions 41 and 42 are provided at positions that cover the second electrode 223 and the third electrode 224 when the cuff cover 4 is mounted to the biological information measurement device 100. Thus, when the biological information measurement device 100 to which the cuff cover 4 is mounted is attached to a human body, the human body and the second electrode 223 and the third electrode 224 can be electrically connected through the conductive fiber portions 41 and 42 of the inner covering portion 10a. In addition, since the conductive fiber portion 41 and the conductive fiber portion 42 are separated from each other by a non-conductive cloth, the second electrode 223 and the third electrode 224 are not short-circuited.

According to such a configuration of the modified example, since the sensing cuff portion 220 together with the electrodes can be covered, the cuff structure can be more reliably protected, and the electrodes and the human body can be electrically connected through the conductive fiber portions.

### Modified Example 2

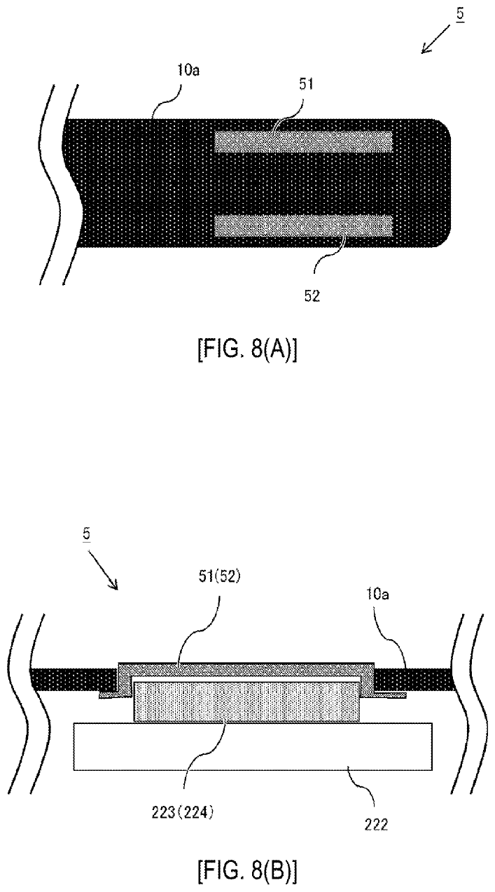

Next, another modified example of the cuff cover will be described. FIG. 8(A) is a partial plan view of the inner covering portion 10a of a cuff cover 5 according to the present modified example, and FIG. 8(B) is an explanatory diagram illustrating conductive cap portions 51 and 52 of the cuff cover 5 according to the present modified example.

Note that since the basic configuration of the cuff cover 5 according to the present modified example is the same as that of the cuff cover 1, such constituent elements are denoted by the same reference signs as those of the cuff cover 1, and redundant description is omitted. As illustrated in FIG. 8(A), the cuff cover 5 according to the present modified example includes the conductive cap portions 51 and 52 at positions corresponding to the opening portions 11 and 12 for electrodes in the inner covering portion 10a.

The conductive cap portions 51 and 52 are formed by sealing opening portions corresponding to the opening portions 11 and 12 for electrodes with lid-shaped members made of a conductive material (for example, a desired metal). Specifically, for example, the lid-shaped member may be formed by thermal welding or the like with a cloth of a peripheral edge portion of the opening portion. Then, as illustrated in FIG. 8(B), the conductive cap portions 51 and 52 are provided at positions that cover the second electrode 223 and the third electrode 224 in the inner covering portion 10a when the cuff cover 5 is mounted to the biological information measurement device 100. Thus, when the biological information measurement device 100 to which the cuff cover 5 is mounted is attached to a human body, the human body can be electrically connected to the second electrode 223 and the third electrode 224 with the conductive cap portions 51 and 52 of the inner covering portion 10a interposed therebetween. Further, since the conductive cap portions 51 and 52 are separated from each other by the non-conductive cloth, the second electrode 223 and the third electrode 224 are not short-circuited.

According to such a configuration of the modified example, since the sensing cuff portion 220 can be covered together with the electrodes, the cuff structure can be more reliably protected, and the electrodes and the human body can be electrically connected through the conductive cap portions.

### Modified Example 3

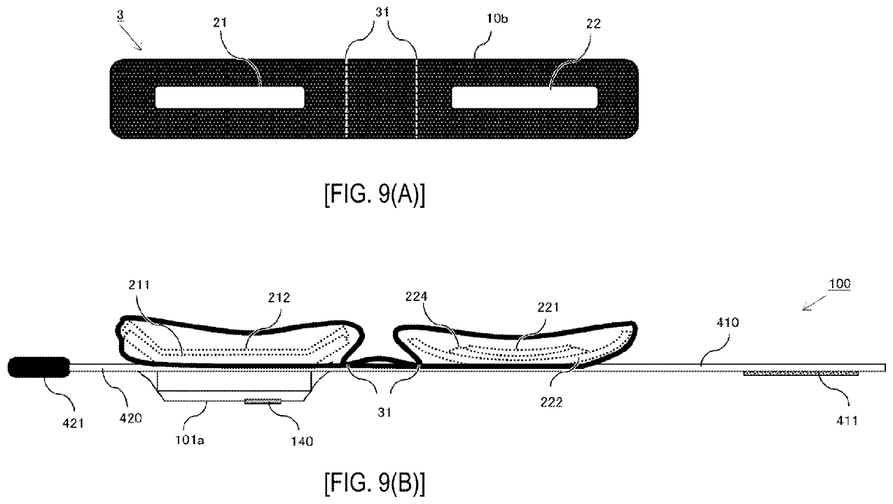

Next, still another modified example of the cuff cover will be described. FIG. 9(A) is a schematic plan view illustrating an appearance of a cuff cover 3 according to the present modified example, and FIG. 9(B) is an explanatory diagram illustrating a state in which the cuff cover 3 according to the present modified example is mounted to the biological information measurement device 100.

Note that since the basic configuration of the cuff cover 3 according to the present modified example is the same as that of the cuff cover 1, such constituent elements are denoted by the same reference signs as those of the cuff cover 1, and redundant description is omitted. As illustrated in FIGS. 9(A) and 9(B), the cuff cover 3 according to the present modified example is provided with a fixing portion 31 configured to fix the inner covering portion 10a and the outer covering portion 10b by, for example, sewing or welding in the vicinity of the center in a longitudinal direction.

As illustrated in FIG. 9(B), when the pressing cuff portion 210 and the sensing cuff portion 220 are accommodated in the covering portion 10, such a fixing portion 31 can eliminate “floating” of the inner covering portion 10a between the pressing cuff portion 210 and the sensing cuff portion 220. This makes it possible to suppress positional deviation between each of the second electrode 223 and the third electrode 224 of the sensing cuff portion 220 accommodated in the covering portion 10 and the conduction assisting portion (the opening portion for electrode, the conductive fiber portion, and the conductive cap portion in the above-described examples) in the inner covering portion 10a.

### Embodiment 2

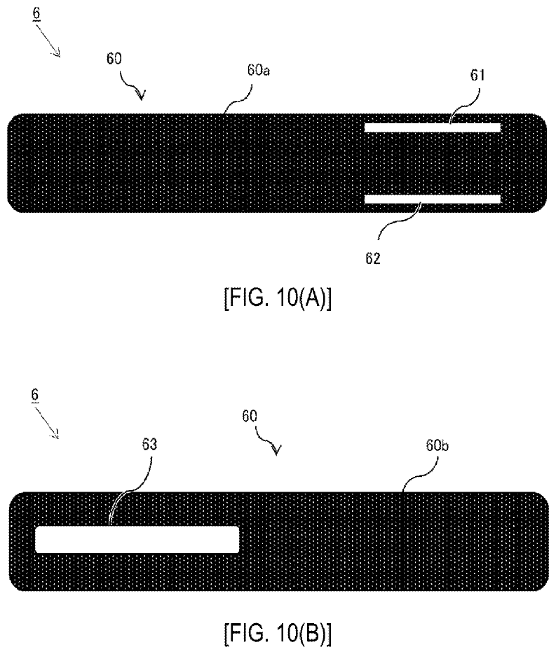

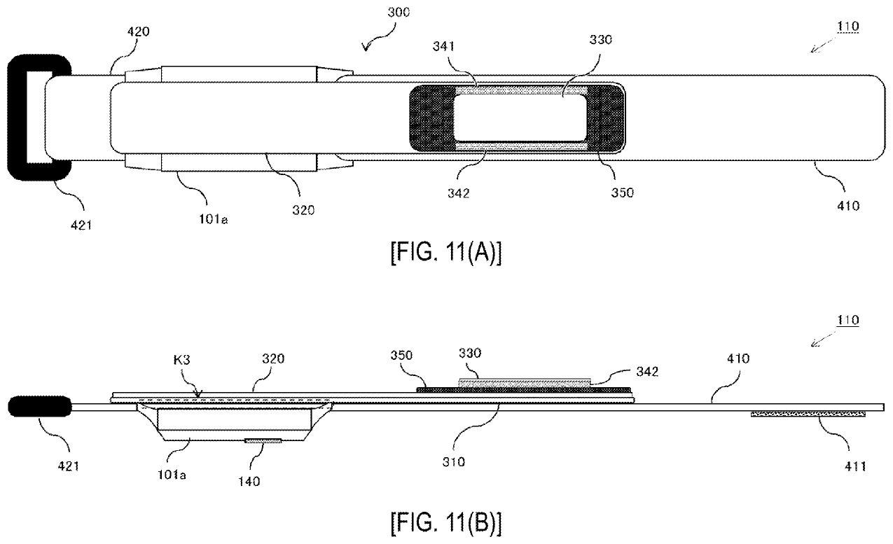

In addition to the above, the cuff cover can have various forms according to the form of the biological information measurement device to which the cuff cover is mounted. Hereafter, another embodiment of the cuff cover according to the present invention will be described based on FIGS. 10(A) and 10(B), and 11(A) and 11(B). FIG. 10(A) is a schematic plan view illustrating an appearance of the cuff cover 6 according to the present embodiment, and FIG. 10(B) is a schematic bottom view illustrating the appearance of the cuff cover 6 according to the present embodiment. FIG. 11(A) is a schematic bottom view illustrating an appearance of the biological information measurement device 110 for which the cuff cover 6 according to the present embodiment is used, and FIG. 11(B) is a schematic side view illustrating the appearance of the biological information measurement device 110 for which the cuff cover 6 according to the present embodiment is used.

As illustrated in FIGS. 10(A) and 10(B), the basic configuration of the cuff cover 6 according to the present embodiment is similar to that of the cuff cover 1 according to Embodiment 1, and a covering portion 60 having a bag shape and including an inner covering portion 60a and an outer covering portion 60b is a main constituent element. Additionally, the cuff cover 6 is also similar to the cuff cover 1 in that opening portions 61 and 62 for electrodes are provided in the inner covering portion 60a. On the other hand, the outer covering portion 60b is different from the cuff cover 1 in that only one accommodation port 63 is present.

Further, as illustrated in FIGS. 11(A) and 11(B), in the present embodiment, the configuration of the cuff structure of the biological information measurement device 110 to which the cuff cover 6 is mounted is different from that of the biological information measurement device 100 according to Embodiment 1. Note that the configurations of the main body unit 101, the belt portion 400, and the like in the biological information measurement device 110 are similar to those in the biological information measurement device 100. Thus, such constituent elements are denoted by the same reference signs as those in Embodiment 1, and will not be described again. The biological information measurement device 110 according to the present embodiment is configured to include only one cuff assembly portion 300 as a configuration related to the cuff structure.

As illustrated in FIGS. 11(A) and 11(B), the cuff assembly portion 300 includes a curler 310, a compression cuff 320, a sensing cuff 330, a second electrode 341, a third electrode 342, and a back plate 350, and these constituent elements are laminated to form an integral structure. The cuff assembly portion 300 also has a longitudinal direction as a whole, and is fixed to the housing 101a of the main body at a fixing portion K3. The cuff cover 6 according to the present embodiment is used to cover such a cuff assembly portion 300.

The curler 310 is a member having a longitudinal direction and serving as a base configured to hold the compression cuff 320, and is disposed on the outermost side (the side away from the wrist) of the cuff assembly portion 300 as illustrated in FIG. 11(B). The compression cuff 320 expands due to the air sent from the pump to tighten the wrist that is an attached portion and apply pressure to the artery (not illustrated) existing in the wrist. Unlike the pressing cuff 211 of the biological information measurement device 100, the compression cuff 320 is not disposed only on the back side of the wrist of the wearer. The compression cuff 320 has a longitudinal direction and extends up to the palm side of the wrist (that is, the outer side of the sensing cuff 330). Other functions of the sensing cuff 330, the back plate 350, the second electrode 341, and the third electrode 342 are substantially the same as those of the corresponding configurations of the biological information measurement device 100 according to Embodiment 1, and thus, description thereof is omitted.

When the cuff cover 6 according to the present embodiment is mounted to the cuff assembly portion 300, for example, the end portion of the cuff assembly portion 300 in the longitudinal direction on the side where the sensing cuff 330 is provided is inserted into the covering portion 60 while pushing and spreading the accommodation port 63 of the cuff cover 6. Then, the cuff assembly portion 300 is inserted along the longitudinal direction of the cuff cover 60, and when the end portion in the longitudinal direction on the side where the sensing cuff 330 is present reaches the end portion inside the covering portion 60, the end portion on the opposite side of the covering portion 60 may be pulled and hooked to the other end portion side of the cuff assembly portion 300. In this way, the accommodation port 63 of the outer covering portion 10b is restored to an opening having a smaller area than an area of the cuff assembly portion 300 in a plan view due to the stretchability of the covering portion 60, so that the accommodation port 63 is hooked by the outer end portion of the cuff assembly portion 300. The cuff cover 6 according to the present embodiment can be mounted to the cuff assembly portion 300 in this manner, and can also be easily detached by performing a procedure reverse to that at the time of being mounted.

### Other Points

The description of the embodiments described above is merely illustrative of the present invention, and the present invention is not limited to the specific embodiments described above. Within the scope of the technical idea of the present invention, various modifications and combinations may be made. For example, it is also possible to apply the configurations of the conduction assisting portion of Modified Examples 1 and 2 of Embodiment 1 described above to Embodiment 2.

In addition, although a configuration in which the covering portion is formed in a bag shape has been described in each of the embodiments and examples described above, the covering portion does not necessarily need to be configured in this manner, and it is also possible to adopt a form in which only the inner surface of the cuff structure is covered with the covering portion formed in a sheet shape.

In addition, the mounting unit of the cuff cover is not limited to the accommodation port of the covering portion formed in the bag shape, and various materials can be used. For example, an adhesive, a double-sided tape, a rubber band, a hook-and-loop fastener, snap buttons, or the like can be employed.

Moreover, in each of the embodiments and examples described above, the cuff structure includes a plurality of electrodes. However, the present invention can also be applied to a cuff cover of a biological information measurement device including a cuff structure including only one electrode.

Further, in each of the embodiments and examples described above, the case where the biological information measurement device 100 is attached to a wrist has been described, but the present invention can also be applied to a cuff cover of a biological information measurement device attached to another part of a human body such as an upper arm.

### REFERENCE SIGNS LIST

1, 3, 4, 5, 6. Cuff cover
10 Covering portion
10a Inner covering portion
10b Outer covering portion
11, 12, 61, 62 Opening portion for electrode
21, 22, 63 Accommodation port
31 Fixing portion
41, 42 Conductive fiber portion
51, 52 Conductive cap portion
100, 110 Biological information measurement device
101 Main body unit
111 Display unit
112 Operation unit
140 First electrode
210 Pressing cuff portion
211 Pressing cuff
212 Pressing cuff plate
220 Sensing cuff portion
221, 330 Sensing cuff
222, 350 Back plate
223, 341 Second electrode
224, 342 Third electrode
300 Cuff assembly portion
310 Curler
320 Compression cuff
400 Belt portion
410 First belt portion
411 Hook-and-loop fastener
420 Second belt portion
421 Belt loop
K1, K2, K3 Fixing portion
T Wrist

What is claimed is:

1. A cuff cover configured to cover a cuff structure in a biological information measurement device, the biological information measurement device including the cuff structure including one or more cuffs and one or more electrodes, and an attachment unit, the biological information measurement device being configured to measure a blood pressure value and an electrocardiographic waveform of a human body in a state of being attached to the human body by the attachment unit, the cuff cover comprising:
a covering portion formed of a non-conductive material, the covering portion being configured to cover the cuff structure;
a mounting portion configured to mount the covering portion to the cuff structure; and
an energization assisting unit provided in the covering portion, the energization assisting unit being configured to assist energization between each of the one or more electrodes included in the cuff structure covered with the covering portion and the human body when the biological information measurement device is attached to the human body,
wherein the covering portion is formed of a material having stretchability in a bag shape capable of accommodating the cuff structure, includes a plurality of the accommodation ports that are openings each of which is configured to accommodate the cuff structure in the covering portion, an inner covering portion configured to be brought into contact with the human body in a state in which the cuff structure is accommodated, the inner covering portion being provided with the energization assisting unit, an outer covering portion configured to be opposed to the inner covering portion with the cuff structure interposed between the outer covering portion and the inner covering portion in the state in which the cuff structure is accommodated, and a fixing portion configured to fix the inner covering portion and the outer covering portion at a position between one of the plurality of the accommodation ports and another of the plurality of the accommodation ports, and is configured to accommodate a plurality of structures including the cuff structure in the covering portion from different accommodation ports,
the accommodation port is provided in the outer covering portion, is an opening having an area smaller than an area of the cuff structure in a plan view of the cuff structure, and is expandable to be larger than the area of the cuff structure due to the stretchability of the material when the cuff structure is accommodated,
the mounting unit is the accommodation port in the covering portion,
the energization assisting unit is a conductive portion configured to cover a portion including each of the one or more electrodes included in the cuff structure in a state where the cuff structure is covered with the covering portion, and
the conductive portion is formed by covering at least a part of an opening provided in the covering portion with a lid-shaped member made of a conductive material.

2. A cuff cover configured to cover a cuff structure in a biological information measurement device, the biological information measurement device including the cuff structure including one or more cuffs and one or more electrodes, and an attachment unit, the biological information measurement device being configured to measure a blood pressure value and an electrocardiographic waveform of a human body in a state of being attached to the human body by the attachment unit, the cuff cover comprising:
a covering portion formed of a non-conductive material, the covering portion being configured to cover the cuff structure;
a mounting portion configured to mount the covering portion to the cuff structure; and
an energization assisting unit provided in the covering portion, the energization assisting unit being configured to assist energization between each of the one or more electrodes included in the cuff structure covered with the covering portion and the human body when the biological information measurement device is attached to the human body,
wherein the covering portion is formed of a material having stretchability in a bag shape capable of accommodating the cuff structure, includes a plurality of the accommodation ports that are openings each of which is configured to accommodate the cuff structure in the covering portion, is configured to accommodate a plurality of structures including the cuff structure in the covering portion from different accommodation ports, includes an inner covering portion configured to be brought into contact with the human body in a state in which the cuff structure is accommodated, the inner covering portion being provided with the energization assisting unit, and an outer covering portion configured to be opposed to the inner covering portion with the cuff structure interposed between the outer covering portion and the inner covering portion in the state in which the cuff structure is accommodated, and is provided with a fixing portion configured to fix the inner covering portion and the outer covering portion at a position between one of the plurality of the accommodation ports and another of the plurality of the accommodation ports,
the accommodation port is provided in the outer covering portion, is an opening having an area smaller than an area of the cuff structure in a plan view of the cuff structure, and is expandable to be larger than the area of the cuff structure due to the stretchability of the material when the cuff structure is accommodated, and
the accommodation port is the mounting unit.
